(19) United States
(12) Patent Application Publication
(10) Pub. No.: US 2014/0243299 A1
(43) Pub. Date: Aug. 28, 2014

(54) Topical Formulations of Corticosteroids with Enhanced Bioavailability

(71) Applicant: Precision Dermatology, Inc., Cumberland, RI (US)

(72) Inventors: Ronald M. Gurge, Franklin, MA (US); Mark W. Trumbore, Westford, MA (US); Pinaki R. Majhi, Sharon, MA (US)

(73) Assignee: Precision Dermatology, Inc., Cumberland, RI (US)

(21) Appl. No.: 14/193,066

(22) Filed: Feb. 28, 2014

Related U.S. Application Data
(60) Provisional application No. 61/770,562, filed on Feb. 28, 2013.

(51) Int. Cl. A61K 9/12; A61K 31/573

(52) U.S. Cl. CPC A61K 9/122; A61K 31/573
USPC 514/179

(57) ABSTRACT

Described herein are methods and compositions for increasing the bioavailability of a corticosteroid, such as hydrocortisone 17-butyrate, in a topical formulation.

### RELATED APPLICATIONS

This application claims the benefit of priority to U.S. Provisional Patent Application Ser. No. 61/770,562, filed Feb. 28, 2013; the contents of which are hereby incorporated by reference.

### BACKGROUND

Currently available topical treatments for inflammatory skin disorders, such as psoriasis and atopic dermatitis, are based on a limited number of active ingredients in a narrow range of dosage forms. In the treatment of mild and localized psoriasis, topical corticosteroids remain the drug of choice, although non-steroidal actives, such as retinoids, vitamin D analogs, tars, anthralin, and keratolytics, are also used. In the treatment of atopic dermatitis, corticosteroids are again the treatment of choice, although alternatives include calcineurin inhibitors or the concomitant use of a corticosteroid and a calcineurin inhibitor.

Mineral oils and vegetable oils are commonly used excipients in the oil phases of emulsion-based topical formulations. Although both classes of compounds are oils, their chemistries are fundamentally different. Vegetable oils are complex molecules with both hydrophilic and hydrophobic characteristics; in addition, they are heterodisperse (i.e., they comprise a range of individual fatty acids). In contrast, mineral oils, while still heterogeneous with respect to molecular structure, are much less complex; mineral oils are almost exclusively hydrophobic, and they primarily comprise alkyl chains.

Similarly, surfactants and co-surfactants are commonly used excipients in emulsion based topical formulations. They are used together to tailor emulsion droplet size and emulsion stability. Variation in co-surfactant/surfactant ratios is typically used to maximize formulation stability.

While oil-in-water emulsion-based topical formulations are known, the use of formulation to specifically optimize active ingredient bioavailability and hence therapeutic outcome is not taught. For example, U.S. Pat. No. 5,635,497 teaches oil-in-water emulsion compositions with a high weight fraction of the discontinuous oil phase. However U.S. Pat. No. 5,635,497 does not teach the use of vegetable oils to optimize active ingredient bioavailability and does not teach how the oil phase components and their ratios can be adjusted to optimize therapeutic outcomes.

U.S. Pat. Nos. 7,378,405, 7,981,877, 8,399,502 and 8,546,364 teach oil-in-water emulsion formulations containing vegetable oils with high linoleic acid content. These patents teach the use of the vegetable oil as a chemical stabilizing agent for the incorporated active ingredient. None of these patents teaches the use of vegetable oils to optimize active ingredient bioavailability, or how the oil phase components and their ratios can be adjusted to optimize therapeutic outcomes.

US patent application publication 2011/0305643 teaches oil-in-water emulsion-based aerosol foam compositions containing high weight percentages of oil phases. Although the compositions disclosed in US 2011/0305643 contain vegetable oils, the published application does not teach the use of vegetable oils to optimize active ingredient bioavailability, nor does it teach adjusting the oil phase components and their ratios to optimize therapeutic outcomes.

There exists a need for methods of formulating topical formulations, wherein the bioavailability of active ingredients is optimized, and can be precisely and accurately predicted.

### SUMMARY OF THE INVENTION

In certain embodiments, the invention relates to a method for enhancing the bioavailability of a corticosteroid from an oil-in-water emulsion, comprising the step of varying the concentrations of surfactants, co-surfactants, emollients and water, thereby forming an improved corticosteroid-containing emulsion.

In certain embodiments, the invention relates to the aforementioned method, wherein the improved corticosteroid-containing emulsion comprises

a corticosteroid;

a surfactant and a co-surfactant;

an oil phase comprising at least a first emollient and a second emollient; and

water;

wherein the first emollient is a vegetable oil and the second emollient is a mineral oil; and the weight ratio of vegetable oil-to-mineral oil is about 0.03 to about 1.00.

In certain embodiments, the invention relates to any one of the aforementioned methods, wherein the corticosteroid is hydrocortisone 17-butyrate (HCB).

In certain embodiments, the invention relates to a method of treating a skin disorder, comprising the step of:

applying topically to an area of skin of a subject in need thereof a therapeutically-effective amount of any one of the aforementioned improved corticosteroid-containing emulsions.

### BRIEF DESCRIPTION OF THE FIGURES

FIG. 1 tabulates the weight percentages of the components of various exemplary formulations of the invention. *N.P.=not present.

FIG. 2 tabulates demographic information for the patient population (ITT population) in the vasoconstriction assays described in Example 2.

FIG. 3 tabulates a summary of vasoconstriction scores (ITT population). *Treatments with the same letter (A-E) are not significantly different from each other. †Grouping based on the REGWQ of the mean scores.

FIG. 4 depicts a histogram of vasoconstriction visual score sums (ITT population).

FIG. 5 depicts a histogram of vasoconstriction mean visual scores (ITT population).

FIG. 6 tabulates data on in vitro release of hydrocortisone butyrate from various exemplary formulations of the invention.

FIG. 7 depicts the cumulative amount of hydrocortisone 17-butyrate (“hydrocortisone butyrate”) released as a function of time for various exemplary formulations of the invention.

FIG. 8 depicts the cumulative amount of hydrocortisone 17-butyrate (“hydrocortisone butyrate”) released as a function of time for various exemplary formulations of the invention.

FIG. 9 depicts the rate of hydrocortisone 17-butyrate (“hydrocortisone butyrate”) release as a function of time for various exemplary formulations of the invention.

FIG. 10 depicts the rate of hydrocortisone 17-butyrate (“hydrocortisone butyrate”) release as a function of time for various exemplary formulations of the invention.

FIG. 11 tabulates the densities of various exemplary foam formulations of the invention.

FIG. 12 tabulates the viscosities of various exemplary formulations of the invention.

FIG. 13 tabulates demographic information for the patient population (ITT population) in the clinical efficacy trial described in Example 6.

FIG. 14 depicts the average percent decrease in Atopic Dermatitis involved Body Surface Area for exemplary formulations of the invention as a function of treatment time. Left bar=vehicle; middle bar=0.1% HCB; right bar=0.15% HCB.

FIG. 15 depicts the percentage of the treatment population exhibiting improvement in Lichenification symptoms after 29 days of treatment with exemplary formulations of the invention. Left bar=vehicle; middle bar=0.1% HCB; right bar=0.15% HCB.

FIG. 16 depicts the percentage of the treatment population exhibiting improvement in Excoriation after 29 days of treatment with exemplary formulations of the invention. Left bar=vehicle; middle bar=0.1% HCB; right bar=0.15% HCB.

FIG. 17 depicts the percentage of the treatment population exhibiting improvement in Oozing/Crusting symptoms after 15 days of treatment with exemplary formulations of the invention. Left bar=vehicle; middle bar=0.1% HCB; right bar=0.15% HCB.

FIG. 18 depicts the percentage of the treatment population exhibiting improvement in Induration/Papulation after 15 days of treatment with exemplary formulations of the invention. Left bar=vehicle; middle bar=0.1% HCB; right bar=0.15% HCB.

### DETAILED DESCRIPTION OF THE INVENTION

### Overview

In certain embodiments, the invention relates to a method for enhancing the bioavailability of a topical corticosteroid by formulating the active ingredient in a high viscosity oil-in-water emulsion containing greater than 30% oil phase components and less than 70% water, then packaging into aerosol cans and pressurizing with hydrofluorocarbon propellants. When the aerosol can is actuated a dense time- and temperature-stable foam is dispensed. In certain embodiments, the invention relates to a dispensed foam that contains a corticosteroid, such as hydrocortisone butyrate, and is suitable for the topical treatment of inflammatory skin disorders. In certain embodiments, the dispensed foam has a density between 0.05 and 0.5 g/cm³, is easily spread over large areas of body surface, is time- and temperature-stable, moisturizes the skin, reduces transepidermal water loss, is well-tolerated, is non-irritating, and improves active ingredient bioavailability. In certain embodiments, the foam rapidly collapses when subjected to shear forces, allowing for rapid and efficient application to large areas of body surface. In the treatment of inflammatory skin disorders, the dispensed foam may be applied to affected areas at least once per day.

In certain embodiments, the oil-in-water emulsions that form the aerosol foam concentrates contain about 8.0% to about 12.0% surfactants/co-surfactants, about 20.0% to about 25.5% emollients, and about 54.0% to about 72.0% water. In certain embodiments, more specifically, the aerosol foam concentrate contains about 4.5% to about 7.0% cetostearyl alcohol, about 5.0% to about 7.0% Ceteth-20, about 5.5% to about 6.5% Safflower Oil, about 10.5% to about 11.5% Light Mineral Oil, about 0.85% to about 0.95% Dimethicone, and about 6.0% to about 7.0% white petrolatum. In certain embodiments, the aerosol foam concentrate compositions have viscosities from about 55,000 to about 110,000 cps. In certain embodiments, the densities of the compositions defined by the method are about 0.13 to about 0.50 g/cm³. In certain embodiments, the aerosol foam compositions of the method exhibit mean vasoconstrictor assay (VCA) scores from about 0.9 to about 1.5.

In certain embodiments, the invention relates to simultaneous systematic variation in the ratios of vegetable and mineral oils and co-surfactant/surfactant ratios to achieve a stated goal. In certain embodiments, the invention relates to the optimization of the bioavailability of active ingredients from topical formulations, which in turn allows for optimization of therapeutic outcomes.

### DEFINITIONS

For convenience, certain terms employed in the specification and appended claims are collected here. These definitions should be read in light of the entire disclosure and understood as by a person of skill in the art.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The phrase “or,” as used herein in the specification and in the claims, should be understood to mean “either or both” of the elements so conjoined, i.e., elements that are conjunctively present in some cases and disjunctively present in other cases. Multiple elements listed with “or” should be construed in the same fashion, i.e., “one or more” of the elements so conjoined. Other elements may optionally be present other than the elements specifically identified by the “or” clause, whether related or unrelated to those elements specifically identified. Thus, as a non-limiting example, a reference to “A or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### Exemplary Constituents of Emulsions and Compositions of the Invention

Exemplary identities of various constituents of the compositions of the present invention are described below.

### 1. Propellants

In certain embodiments, the propellant is a HFA or a mixture of one or more hydrofluorocarbons. Suitable hydrofluorocarbons include 1,1,1,2-tetrafluoroethane (HFA 134a); 1,1,1,2,3,3,3-heptafluoropropane (HFA 227); and mixtures and admixtures of these and other HFAs that are currently approved or may become approved for medical use are suitable. The concentration of the HFA propellant is about 2% to about 50% by weight of the composition. In certain embodiments, the propellant comprises a hydrofluoroolefin (HFO), or a mixture of HFO and HFA. Suitable hydrofluoroolefins include 1,3,3,3-tetrafluoropropene (HFO 1234ze) and mixtures and admixtures of this and other HFO suitable for topical use. The concentration of the HFO propellant is about 2% to about 50% by weight of the composition. Hydrocarbon as well as CFC propellants can also be used in the present invention.

### 2. Vehicles

Suitable topical vehicles and vehicle components for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerol (glycerin)), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, dimethiconol, dimethicone, and dimethicone copolyol; hydrocarbon-based materials such as petrolatum and squalane; and other vehicles and vehicle components that are suitable for administration to the skin, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In one embodiment, formulations without methanol, ethanol, propanols, or butanols are desirable.

### 3. Surfactants and Emulsifiers

Many topical formulations contain chemical emulsions which use surface active ingredients (emulsifiers and surfactants) to disperse dissimilar chemicals in a particular solvent system. For example, most lipid-like (oily or fatty) or lipophilic ingredients do not uniformly disperse in aqueous solvents unless they are first combined with emulsifiers, which form microscopic aqueous soluble structures (droplets) that contain a lipophilic interior and a hydrophilic exterior, resulting in an oil-in-water emulsion. In order to be soluble in aqueous media, a molecule must be polar or charged so as to favorably interact with water molecules, which are also polar. Similarly, to dissolve an aqueous-soluble polar or charged ingredient in a largely lipid or oil-based solvent, an emulsifier is typically used which forms stable structures that contain the hydrophilic components in the interior of the structure while the exterior is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion droplet. Emulsion destabilization results in the aqueous and lipophilic ingredients separating into two layers, potentially destroying the commercial value of a topical product.

Surfactants suitable for use in the present invention may be ionic or non-ionic. These include, but are not limited to: sodium isostearate, cetyl alcohol, polysorbates (Polysorbate 20, Polysorbate 40, Polysorbate 60, Polysorbate 80), steareth-10 (Brij 76), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate (dihexadecyl phosphate), ceteareth-10 phosphate, methylbenzethonium chloride, dicetyl phosphate, ceteth-10 phosphate (ceteth-10 is the polyethylene glycol ether of cetyl alcohol where n has an average value of 10; ceteth-10 phosphate is a mixture of phosphoric acid esters of ceteth-10), ceteth-20, Brij S10 (polyethylene glycol octadecyl ether, average Mₙ˜711), PEG-20 phytosterol, and Poloxamers (including, but not limited to, Poloxamer 188 (HO(C₂H₄O)ₐ(CH(CH₃)CH₂O)_{b}(C₂H₄O)ₐH, average molecular weight 8400) and Poloxamer 407 (HO(C₂H₄O)ₐ(CH(CH₃)CH₂O)_{b}(C₂H₄O)ₐH, wherein a is about 101 and b is about 56)). Appropriate combinations or mixtures of such surfactants may also be used according to the present invention.

Many of these surfactants may also serve as emulsifiers in formulations of the present invention.

Other suitable emulsifiers for use in the formulations of the present invention include, but are not limited to, glycine soja protein, sodium lauroyl lactylate, polyglyceryl-4 diisostearate-polyhydroxystearate-sebacate, behentrimonium methosulfate-cetearyl alcohol, non-ionic emulsifiers like emulsifying wax, polyoxyethylene oleyl ether, PEG-40 stearate, carbomer, cetostearyl alcohol (cetearyl alcohol), ceteareth-12, ceteareth-20, ceteareth-25, ceteareth-30, ceteareth alcohol, Ceteth-20 (Ceteth-20 is the polyethylene glycol ether of cetyl alcohol where n has an average value of 20), oleic acid, oleyl alcohol, glyceryl stearate, PEG-75 stearate, PEG-100 stearate, and PEG-100 stearate, ceramide 2, ceramide 3, stearic acid, cholesterol, laureth-12, steareth-2, and steareth-20, or combinations/mixtures thereof, as well as cationic emulsifiers like stearamidopropyl dimethylamine and behentrimonium methosulfate, or combinations/mixtures thereof.

### 4. Moisturizers, Emollients, and Humectants

One of the most important aspects of topical products in general, and cosmetic products in particular, is the consumer's perception of the aesthetic qualities of a product. For example, while white petrolatum is an excellent moisturizer and skin protectant, it is rarely used alone, especially on the face, because it is greasy, sticky, does not rub easily into the skin and may soil clothing. Consumers highly value products which are aesthetically elegant and have an acceptable tactile feel and performance on their skin.

Suitable moisturizers for use in the formulations of the present invention include, but are not limited to, lactic acid and other hydroxy acids and their salts, glycerol, propylene glycol, butylene glycol, sodium PCA, sodium hyaluronate, Carbowax 200, Carbowax 400, and Carbowax 800.

Suitable emollients or humectants for use in the formulations of the present invention include, but are not limited to, panthenol, cetyl palmitate, glycerol (glycerin), PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma grandiflorum seed butter, sunflower seed oil, ceramides (e.g., ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopherylsuccinoylamido hydroxypropane, urea, aloe, allantoin, glycyrrhetinic acid, safflower oil, oleyl alcohol, oleic acid, stearic acid, dicaprylate/dicaprate, diethyl sebacate, isostearyl alcohol, pentylene glycol, isononyl isononanoate, polyquaternium-10 (quaternized hydroxyethyl cellulose), camellia oleifera leaf extract, phytosteryl canola glycerides, shea butter, caprylic/capric triglycerides, punica granatum sterols, ethylhexyl stearate, betaine, behenyl alcohol (docosanol), stearyl alcohol (1-octadecanol), laminaria ochroleuca extract, behenic acid, caproyl sphingosine, caproyl phytosphingosine, dimethicone-divinyldimethicone-silsesquioxane crosspolymer, potassium lactate, sodium hyaluronate crosspolymer, hydrolyzed hyaluronic acid, sodium butyroyl-formoyl hyaluronate, polyglutamic acid, tetradecyl aminobutyroylvalylaminobutyric urea trifluoroacetate, micrococcus lysate, hydrolyzed rice bran protein, glycine soja protein, and 1,3-bis(N-2-(hydroxyethyl)palmitoylamino)-2-hydroxypropane.

In addition, appropriate combinations and mixtures of any of these moisturizing agents and emollients may be used in accordance with the present invention. Many of these are classified as “skin conditioners.”

### 5. Preservatives and Antioxidants

The composition may further include components adapted to improve the stability or effectiveness of the applied formulation.

Suitable preservatives for use in the present invention include, but are not limited to: ureas, such as imidazolidinyl urea and diazolidinyl urea; chlorphenesin; methylisothiazolinone; phenoxyethanol; sodium methyl paraben, methylparaben, ethylparaben, and propylparaben; ethylhexyl glycerin; potassium sorbate; sodium benzoate; sorbic acid; benzoic acid; caprylyl glycol; formaldehyde; phytosphingosine; citric acid; sodium citrate; zinc citrate; chlorine dioxide; quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; piroctone olamine; Vitis vinifera seed oil; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, butylated hydroxyanisole, tocopherols (such as α-tocopherol), tocopheryl acetate, superoxide dismutase, oxidoreductases, Arabidopsis thaliana extract, chrysin, black raspberry seed oil, raspberry seed oil, pomegranate seed oil, cranberry seed oil, sodium ascorbate/ascorbic acid, ascorbyl palmitate, propyl gallate, and chelating agents like EDTA (e.g., disodium EDTA), citric acid, and sodium citrate.

In certain embodiments, the antioxidant or preservative comprises (3-(4-chlorophenoxy)-2-hydroxypropyl)carbamate.

In certain embodiments, antioxidants or preservatives of the present invention may also function as a moisturizer or emollient, for example.

In addition, combinations or mixtures of these preservatives or anti-oxidants may also be used in the formulations of the present invention.

### 6. Active Agents

The active agent may be any material that has a desired effect when applied topically to a mammal, particularly a human. Suitable classes of active agents include, but are not limited to, antibiotic agents, antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones. Mixtures of any of these active agents may also be employed. Additionally, dermatologically-acceptable salts and esters of any of these agents may be employed.

6.1 Antibiotics

Representative antibiotics include, without limitation, benzoyl peroxide, alfa terpineol, octopirox, erythromycin, zinc, tetracyclin, triclosan, azelaic acid and its derivatives, phenoxy ethanol and phenoxy propanol, ethyl acetate, clindamycin (e.g., clindamycin phosphate) and meclocycline; sebostats such as flavinoids; alpha and beta hydroxy acids; and bile salts such as scymnol sulfate and its derivatives, deoxycholate and cholate. The antibiotic can be an antifungal agent. Suitable antifungal agents include, but are not limited to, clotrimazole, econazole, ketoconazole, itraconazole, miconazole, oxiconazole, sulconazole, butenafine, naftifine, terbinafine, undecylinic acid, tolnaftate, and nystatin. Mixtures of these antibiotic agents may also be employed. Additionally, dermatologically-acceptable salts and esters of any of these agents may be employed.

6.2 Non-Steroidal Anti-Inflammatory Agents

Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac, fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone; and niacinamide. Mixtures of these non-steroidal anti-inflammatory agents may also be employed, as well as the dermatologically acceptable salts and esters of these agents. For example, etofenamiate, a flufenamic acid derivative, is particularly useful for topical application.

6.3 Steroidal Anti-Inflammatory Agents

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters (including betamethasone dipropionate), chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

6.4 Anesthetics

Suitable anesthetics include the aminoacylanilide compounds such as lidocaine, prilocaine, bupivacaine, levo-bupivacaine, ropivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, butamben, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amide compounds such as dibucaine and related local anesthetic compounds; aminoketone compounds such as falicaine, dyclonine and related local anesthetic compounds; and amino ether compounds such as pramoxine, dimethisoquien, and related local anesthetic compounds; and para-amino benzoic acid esters such as benzocaine. Other suitable local anesthetics include ketocaine, dibucaine, amethocaine, propanacaine, and propipocaine.

6.5 Antimicrobial Agents

Suitable antimicrobial agents include, but are not limited to, antibacterial, antifungal, antiprotozoal and antiviral agents, such as beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin (e.g., clindamycin phosphate), ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, famesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, clindamycin phosphate, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, nystatin, tolnaftate, clotrimazole, anidulafungin, micafungin, voriconazole, lanoconazole, ciclopirox and mixtures thereof.

6.6 Keratolytic Agents

Suitable keratolytic agents include, but are not limited to, urea, salicylic acid, papain, bromelain, sulfur, glycolic acid, pyruvic acid, resorcinol, N-acetylcysteine, mandelic acid, retinoids such as retinoic acid (e.g., tretinoin) and its derivatives (e.g., cis and trans, esters), retinol, alpha hydroxy acids, beta hydroxy acids, coal tar, and combinations thereof.

### 7. Purging Gases

In one embodiment, the air in the container charged with the composition is replaced by an inert gas. In certain embodiments, the inert gas is selected from the group consisting of argon, nitrogen, and mixtures thereof.

### 8. Buffer Salts

Suitable buffer salts are well-known in the art. Examples of suitable buffer salts include, but are not limited to sodium citrate, citric acid, sodium phosphate monobasic, sodium phosphate dibasic, sodium phosphate tribasic, potassium phosphate monobasic, potassium phosphate dibasic, and potassium phosphate tribasic.

### 9. Viscosity Modifiers

Suitable viscosity adjusting agents (i.e., thickening and thinning agents or viscosity modifying agents) for use in the formulations of the present invention include, but are not limited to, protective colloids or non-ionic gums such as hydroxyethylcellulose, xanthan gum, and sclerotium gum, as well as magnesium aluminum silicate, silica, microcrystalline wax, beeswax, paraffin, and cetyl palmitate. Crosspolymers of acrylates/C₁₀₋₃₀ alkyl acrylate are also considered. In addition, appropriate combinations or mixtures of these viscosity adjusters may be utilized according to the present invention.

### 10. Additional Constituents

Additional constituents suitable for incorporation into the emulsions of the present invention include, but are not limited to: skin protectants, adsorbents, demulcents, emollients, moisturizers, sustained release materials, solubilizing agents, skin-penetration agents, skin soothing agents, deodorant agents, antiperspirants, sun screening agents, sunless tanning agents, vitamins, hair conditioning agents, anti-irritants, anti-aging agents, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, lipids, immunomodulators, and pH adjusters (e.g., citric acid, sodium hydroxide, and sodium phosphate).

For example, lipids normally found in healthy skin (or their functional equivalents) may be incorporated into the emulsions of the present invention. In certain embodiments, the lipid is selected from the group consisting of ceramides, cholesterol, and free fatty acids. Examples of lipids include, but are not limited to, ceramide 1, ceramide 2, ceramide 3, ceramide 4, ceramide 5, ceramide 6, hydroxypropyl bispalmitamide MEA, and hydroxypropyl bislauramide MEA, and combinations thereof.

Examples of peptides that interact with protein structures of the dermal-epidermal junction include palmitoyl dipeptide-5 diaminobutyloyl hydroxythreonine, palmitoyl tripeptide-5, acetyl octapeptide-3, pentapeptide-3, palmitoyl dipeptide-5 diaminohydroxybutyrate, dipeptide diaminobutyroyl benzylamide diacetate, palmitoyl tetrapeptide-7, palmitoyl oligopeptide, and palmitoyl dipeptide-6 diaminohydroxybutyrate.

Examples of skin soothing agents include, but are not limited to algae extract, mugwort extract, stearyl glycyrrhetinate, bisabolol, allantoin, aloe, avocado oil, green tea extract, hops extract, chamomile extract, colloidal oatmeal, calamine, cucumber extract, and combinations thereof.

N-hydroxysuccinimide activates the elimination of blood originated pigments responsible for dark color and inflammation that causes under eye circles.

In certain embodiments, the compositions comprise bergamot or bergamot oil. Bergamot oil is a natural skin toner and detoxifier. In certain embodiments, it may prevent premature aging of skin and may have excellent effects on oily skin conditions and acne.

Examples of vitamins include, but are not limited to, vitamins A, D, E, K, and combinations thereof. Vitamin analogues are also contemplated; for example, the vitamin D analogues calcipotriene or calcipotriol.

In certain embodiments, the vitamin may be present as tetrahexyldecyl ascorbate. This compound exhibits anti-oxidant activity, inhibiting lipid peroxidation. In certain embodiments, use can mitigate the damaging effects of UV exposure. Studies have shown it to stimulate collagen production as well as clarifying and brightening the skin by inhibiting melanogenesis (the production of pigment) thereby promoting a more even skin tone.

Examples of sunscreens include, but are not limited to, p-aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, zinc oxide, 4-methylbenzylidene camphor, methylene bis-benzotriazolyl tetramethylbutylphenol, bis-ethylhexyloxyphenol methoxyphenyl triazine, terephthalylidene dicamphor sulfonic acid, drometrizole trisiloxane, disodium phenyl dibenzimidazole tetrasulfonate, diethylamino hydroxybenzoyl hexyl benzoate, octyl triazone, diethylhexyl butamido triazone, polysilicone-15, and combinations thereof.

Suitable fragrances and colors may be used in the formulations of the present invention. Examples of fragrances and colors suitable for use in topical products are known in the art.

Suitable immunomodulators include, but are not limited to, tetrachlorodecaoxide, deoxycholic acid, tacrolimus, pimecrolimus, and beta-glucan.

In certain embodiments, palmitoyl-lysyl-valyl-lysine bistrifluoroacetate is added. This peptide stimulates collagen synthesis in human fibroblasts.

In certain embodiments, plant extracts may be included. Examples include artemisia vulgaris extract, plankton extract, chlorella vulgaris extract, and phytosterol.

An example of a film-forming agent is polysilicone-11.

Often, one constituent of a composition may accomplish several functions. In one embodiment, the present invention relates to constituents that may act as a lubricant, an emollient, or a skin-penetrating agent. In one embodiment, the multi-functional constituent is socetyl stearate, isopropyl isostearate, isopropyl palmitate, or isopropyl myristate.

### Exemplary Oil-in-Water Emulsions and Formulations of the Invention

In certain embodiments, the invention relates to an oil-in-water emulsion, wherein the oil-in-water emulsion comprises

a corticosteroid;

a surfactant and a co-surfactant;

an oil phase comprising at least a first emollient and a second emollient; and

water;

wherein the first emollient is a vegetable oil and the second emollient is a mineral oil; and the weight ratio of vegetable oil-to-mineral oil is about 0.03 to about 1.00.

In certain embodiments, the invention relates to any one of the aforementioned oil-in-water emulsions, wherein the total concentration of surfactants and co-surfactants is about 8.0% to about 12.0% by weight of the emulsion.

In certain embodiments, the invention relates to any one of the aforementioned oil-in-water emulsions, wherein the surfactant is ceteth-20; and the co-surfactant is cetostearyl alcohol.

In certain embodiments, the invention relates to any one of the aforementioned oil-in-water emulsions, wherein the emulsion does not comprise steareth-10.

In certain embodiments, the invention relates to any one of the aforementioned oil-in-water emulsions, wherein the concentration of ceteth-20 is about 5.0% to about 7.0% by weight of the emulsion.

In certain embodiments, the invention relates to any one of the aforementioned oil-in-water emulsions, wherein the concentration of cetostearyl alcohol is about 4.5% to about 7.0% by weight of the emulsion.

In certain embodiments, the invention relates to any one of the aforementioned oil-in-water emulsions, wherein the oil phase comprises a plurality of emollients; and the total concentration of emollients is about 20.0% to about 25.5% by weight of the emulsion.

In certain embodiments, the invention relates to any one of the aforementioned oil-in-water emulsions, wherein the emollients are safflower oil, dimethicone, light mineral oil, and white petrolatum.

In certain embodiments, the invention relates to any one of the aforementioned oil-in-water emulsions, wherein the concentration of safflower oil is about 5.5% to about 6.5% by weight of the emulsion.

In certain embodiments, the invention relates to any one of the aforementioned oil-in-water emulsions, wherein the concentration of dimethicone is about 0.85% to about 0.95% by weight of the emulsion.

In certain embodiments, the invention relates to any one of the aforementioned oil-in-water emulsions, wherein the concentration of light mineral oil is about 10.5% to about 11.5% by weight of the emulsion.

In certain embodiments, the invention relates to any one of the aforementioned oil-in-water emulsions, wherein the concentration of white petrolatum is about 6.0% to about 7.0% by weight of the emulsion.

In certain embodiments, the invention relates to any one of the aforementioned oil-in-water emulsions, wherein the concentration of water is about 54.0% to about 72.0% by weight of the emulsion.

In certain embodiments, the invention relates to any one of the aforementioned oil-in-water emulsions, wherein the corticosteroid is hydrocortisone butyrate.

In certain embodiments, the invention relates to any one of the aforementioned oil-in-water emulsions, wherein the hydrocortisone butyrate is hydrocortisone 17-butyrate.

In certain embodiments, the invention relates to any one of the aforementioned oil-in-water emulsions, wherein the concentration of hydrocortisone-17 butyrate is about 0.1% by weight of the emulsion.

In certain embodiments, the invention relates to any one of the aforementioned oil-in-water emulsions, wherein the concentration of hydrocortisone 17-butyrate is 0.15% by weight of the emulsion.

In certain embodiments, the invention relates to any one of the aforementioned oil-in-water emulsions, wherein the weight ratio of vegetable oil-to-mineral oil is about 0.03, about 0.06, about 0.13, about 0.2, about 0.55, about 0.75, or about 1.00. In certain embodiments, the invention relates to any one of the aforementioned oil-in-water emulsions, wherein the weight ratio of vegetable oil-to-mineral oil is about 0.2 or about 0.55.

In certain embodiments, the invention relates to any one of the aforementioned oil-in-water emulsions, wherein the vegetable oil comprises mono- and poly-unsaturated fatty acids.

In certain embodiments, the invention relates to any one of the aforementioned oil-in-water emulsions, wherein the vegetable oil comprises mono- and poly-unsaturated fatty acids with acyl chain lengths from about 4 to about 28 carbons.

In certain embodiments, the invention relates to any one of the aforementioned oil-in-water emulsions, wherein the vegetable oil comprises poly-unsaturated fatty acids in an amount from about 10% to about 78% by number of fatty acids.

In certain embodiments, the invention relates to any one of the aforementioned oil-in-water emulsions, wherein the poly-unsaturated fatty acid is linoleic acid.

In certain embodiments, the invention relates to any one of the aforementioned oil-in-water emulsions, wherein the vegetable oil is safflower oil, sunflower oil, corn oil, sesame oil, peanut oil, canola oil, or olive oil.

In certain embodiments, the invention relates to any one of the aforementioned oil-in-water emulsions, wherein the vegetable oil is safflower oil.

In certain embodiments, the invention relates to any one of the aforementioned oil-in-water emulsions, wherein the vegetable oil has a viscosity from about 30 cP to about 50 cP at 35° C.

In certain embodiments, the invention relates to any one of the aforementioned oil-in-water emulsions, wherein the vegetable oil has a HLB value from about 6 to about 8. In certain embodiments, the invention relates to any one of the aforementioned oil-in-water emulsions, wherein the vegetable oil has a HLB value of 6, 7, or 8.

In certain embodiments, the invention relates to any one of the aforementioned oil-in-water emulsions, wherein the mineral oil is light mineral oil.

In certain embodiments, the invention relates to any one of the aforementioned oil-in-water emulsions, wherein the mineral oil has a viscosity of about 10 cP to about 20 cP at 35° C.

In certain embodiments, the invention relates to any one of the aforementioned oil-in-water emulsions, wherein the mineral oil has a HLB value of about 9 to about 11. In certain embodiments, the invention relates to any one of the aforementioned oil-in-water emulsions, wherein the mineral oil has a HLB value of 10.

In certain embodiments, the invention relates to any one of the aforementioned oil-in-water emulsions, wherein the oil-in-water emulsion comprises

Hydrocortisone 17-butyrate USP
Water USP
Glycerin USP
Methylparaben NF
Propylparaben NF
Cetostearyl Alcohol NF
Urea USP
Dimethicone NF
Safflower Oil USP
White Petrolatum USP
Light Mineral Oil NF
Ceteth-20 NF
Butylated Hydroxytoluene NF
Sodium Citrate USP
Citric Acid USP

In certain embodiments, the invention relates to any one of the aforementioned oil-in-water emulsions, wherein the oil-in-water emulsion consists essentially of

Hydrocortisone 17-butyrate USP
Water USP
Glycerin USP
Methylparaben NF
Propylparaben NF
Cetostearyl Alcohol NF
Urea USP
Dimethicone NF
Safflower Oil USP
White Petrolatum USP
Light Mineral Oil NF
Ceteth-20 NF
Butylated Hydroxytoluene NF
Sodium Citrate USP
Citric Acid USP

In certain embodiments, the invention relates to any one of the aforementioned oil-in-water emulsions, wherein the oil-in-water emulsion consists of

Hydrocortisone 17-butyrate USP
Water USP
Glycerin USP
Methylparaben NF
Propylparaben NF
Cetostearyl Alcohol NF
Urea USP
Dimethicone NF
Safflower Oil USP
White Petrolatum USP
Light Mineral Oil NF
Ceteth-20 NF
Butylated Hydroxytoluene NF
Sodium Citrate USP
Citric Acid USP

In certain embodiments, the invention relates to any one of the aforementioned oil-in-water emulsions, wherein the oil-in-water emulsion comprises, by weight of the oil-in-water emulsion

| | |
|---|---|
| Hydrocortisone 17-butyrate USP | From about 0.01% to about 0.25% |
| Water USP | From about 30% to about 80% |
| Glycerin USP | From about 2.5% to about 7.5% |
| Methylparaben NF | From about 0.15% to about 0.45% |
| Propylparaben NF | From about 0.05% to about 0.15% |
| Cetostearyl Alcohol NF | From about 2.5% to about 7.5% |
| Urea USP | From about 0.3% to about 0.9% |
| Dimethicone NF | From about 0.5% to about 1.5% |
| Safflower Oil USP | From about 3.0% to about 9.0% |
| White Petrolatum USP | From about 3.0% to about 9.0% |
| Light Mineral Oil NF | From about 6.0% to about 18.0% |
| Ceteth-20 NF | From about 3.0% to about 9.0% |
| Butylated Hydroxytoluene NF | From about 0.015% to about 0.045% |
| Sodium Citrate USP | From about 0.15% to about 0.45% |
| Citric Acid USP | From about 0.20% to about 0.60% |

In certain embodiments, the invention relates to any one of the aforementioned oil-in-water emulsions, wherein the oil-in-water emulsion consists essentially of, by weight of the oil-in-water emulsion

| | |
|---|---|
| Hydrocortisone 17-butyrate USP | From about 0.01% to about 0.25% |
| Water USP | From about 30% to about 80% |
| Glycerin USP | From about 2.5% to about 7.5% |
| Methylparaben NF | From about 0.15% to about 0.45% |
| Propylparaben NF | From about 0.05% to about 0.15% |
| Cetostearyl Alcohol NF | From about 2.5% to about 7.5% |
| Urea USP | From about 0.3% to about 0.9% |
| Dimethicone NF | From about 0.5% to about 1.5% |
| Safflower Oil USP | From about 3.0% to about 9.0% |
| White Petrolatum USP | From about 3.0% to about 9.0% |
| Light Mineral Oil NF | From about 6.0% to about 18.0% |
| Ceteth-20 NF | From about 3.0% to about 9.0% |
| Butylated Hydroxytoluene NF | From about 0.015% to about 0.045% |
| Sodium Citrate USP | From about 0.15% to about 0.45% |
| Citric Acid USP | From about 0.20% to about 0.60% |

In certain embodiments, the invention relates to any one of the aforementioned oil-in-water emulsions, wherein the oil-in-water emulsion consists of, by weight of the oil-in-water emulsion

| | |
|---|---|
| Hydrocortisone 17-butyrate USP | From about 0.01% to about 0.25% |
| Water USP | From about 30% to about 80% |
| Glycerin USP | From about 2.5% to about 7.5% |
| Methylparaben NF | From about 0.15% to about 0.45% |
| Propylparaben NF | From about 0.05% to about 0.15% |
| Cetostearyl Alcohol NF | From about 2.5% to about 7.5% |
| Urea USP | From about 0.3% to about 0.9% |
| Dimethicone NF | From about 0.5% to about 1.5% |
| Safflower Oil USP | From about 3.0% to about 9.0% |
| White Petrolatum USP | From about 3.0% to about 9.0% |
| Light Mineral Oil NF | From about 6.0% to about 18.0% |
| Ceteth-20 NF | From about 3.0% to about 9.0% |
| Butylated Hydroxytoluene NF | From about 0.015% to about 0.045% |
| Sodium Citrate USP | From about 0.15% to about 0.45% |
| Citric Acid USP | From about 0.20% to about 0.60% |

In certain embodiments, the invention relates to any one of the aforementioned oil-in-water emulsions, wherein the oil-in-water emulsion comprises, by weight of the oil-in-water emulsion

| | |
|---|---|
| Hydrocortisone 17-butyrate USP | About 0.1% |
| Water USP | About 56.45% |
| Glycerin USP | About 5.00% |
| Methylparaben NF | About 0.30% |
| Propylparaben NF | About 0.10% |
| Cetostearyl Alcohol NF | About 5.34% |
| Urea USP | About 0.64% |
| Dimethicone NF | About 0.92% |
| Safflower Oil USP | About 6.18% |
| White Petrolatum USP | About 6.87% |
| Light Mineral Oil NF | About 11.33% |
| Ceteth-20 NF | About 6.00% |
| Butylated Hydroxytoluene NF | About 0.03% |
| Sodium Citrate USP | About 0.32% |
| Citric Acid USP | About 0.42% |

In certain embodiments, the invention relates to any one of the aforementioned oil-in-water emulsions, wherein the oil-in-water emulsion consists essentially of, by weight of the oil-in-water emulsion

| | |
|---|---|
| Hydrocortisone 17-butyrate USP | About 0.1% |
| Water USP | About 56.45% |
| Glycerin USP | About 5.00% |
| Methylparaben NF | About 0.30% |
| Propylparaben NF | About 0.10% |
| Cetostearyl Alcohol NF | About 5.34% |
| Urea USP | About 0.64% |
| Dimethicone NF | About 0.92% |
| Safflower Oil USP | About 6.18% |
| White Petrolatum USP | About 6.87% |
| Light Mineral Oil NF | About 11.33% |
| Ceteth-20 NF | About 6.00% |
| Butylated Hydroxytoluene NF | About 0.03% |
| Sodium Citrate USP | About 0.32% |
| Citric Acid USP | About 0.42% |

In certain embodiments, the invention relates to any one of the aforementioned oil-in-water emulsions, wherein the oil-in-water emulsion consists of, by weight of the oil-in-water emulsion

| | |
|---|---|
| Hydrocortisone 17-butyrate USP | About 0.1% |
| Water USP | About 56.45% |
| Glycerin USP | About 5.00% |
| Methylparaben NF | About 0.30% |
| Propylparaben NF | About 0.10% |
| Cetostearyl Alcohol NF | About 5.34% |
| Urea USP | About 0.64% |
| Dimethicone NF | About 0.92% |
| Safflower Oil USP | About 6.18% |
| White Petrolatum USP | About 6.87% |
| Light Mineral Oil NF | About 11.33% |
| Ceteth-20 NF | About 6.00% |
| Butylated Hydroxytoluene NF | About 0.03% |
| Sodium Citrate USP | About 0.32% |
| Citric Acid USP | About 0.42% |

In certain embodiments, the invention relates to any one of the aforementioned oil-in-water emulsions, wherein the oil-in-water emulsion comprises

| | |
|---|---|
| Hydrocortisone 17-butyrate USP | About 0.15% |
| Water USP | About 56.40% |
| Glycerin USP | About 5.00% |
| Methylparaben NF | About 0.30% |
| Propylparaben NF | About 0.10% |
| Cetostearyl Alcohol NF | About 5.34% |
| Urea USP | About 0.64% |
| Dimethicone NF | About 0.92% |
| Safflower Oil USP | About 6.18% |
| White Petrolatum USP | About 6.87% |
| Light Mineral Oil NF | About 11.33% |
| Ceteth-20 NF | About 6.00% |
| Butylated Hydroxytoluene NF | About 0.03% |
| Sodium Citrate USP | About 0.32% |
| Citric Acid USP | About 0.42% |

In certain embodiments, the invention relates to any one of the aforementioned oil-in-water emulsions, wherein the oil-in-water emulsion consists essentially of

| | |
|---|---|
| Hydrocortisone 17-butyrate USP | About 0.15% |
| Water USP | About 56.40% |
| Glycerin USP | About 5.00% |
| Methylparaben NF | About 0.30% |
| Propylparaben NF | About 0.10% |
| Cetostearyl Alcohol NF | About 5.34% |
| Urea USP | About 0.64% |
| Dimethicone NF | About 0.92% |
| Safflower Oil USP | About 6.18% |
| White Petrolatum USP | About 6.87% |
| Light Mineral Oil NF | About 11.33% |
| Ceteth-20 NF | About 6.00% |
| Butylated Hydroxytoluene NF | About 0.03% |
| Sodium Citrate USP | About 0.32% |
| Citric Acid USP | About 0.42% |

In certain embodiments, the invention relates to any one of the aforementioned oil-in-water emulsions, wherein the oil-in-water emulsion consists of

| | |
|---|---|
| Hydrocortisone 17-butyrate USP | About 0.15% |
| Water USP | About 56.40% |
| Glycerin USP | About 5.00% |
| Methylparaben NF | About 0.30% |
| Propylparaben NF | About 0.10% |
| Cetostearyl Alcohol NF | About 5.34% |
| Urea USP | About 0.64% |
| Dimethicone NF | About 0.92% |
| Safflower Oil USP | About 6.18% |
| White Petrolatum USP | About 6.87% |
| Light Mineral Oil NF | About 11.33% |
| Ceteth-20 NF | About 6.00% |
| Butylated Hydroxytoluene NF | About 0.03% |
| Sodium Citrate USP | About 0.32% |
| Citric Acid USP | About 0.42% |

In certain embodiments, the invention relates to a formulation, wherein the formulation comprises, consists essentially of, or consists of any one of the aforementioned oil-in-water emulsions in admixture with a propellant and an inert gas.

In certain embodiments, the invention relates to any one of the aforementioned formulations, wherein the formulation is a foamable formulation.

In certain embodiments, the invention relates to any one of the aforementioned formulations, wherein the formulation is packaged in an aerosol container.

In certain embodiments, the invention relates to any one of the aforementioned formulations, wherein, upon expulsion from the aerosol container, the formulation forms a foam.

In certain embodiments, the invention relates to any one of the aforementioned formulations, wherein the formulation comprises the propellant in an amount from about 8% to about 15% by weight of the formulation.

In certain embodiments, the invention relates to any one of the aforementioned formulations, wherein the formulation comprises the inert gas in an amount from about 0.8% to about 4.0% by weight of the formulation.

In certain embodiments, the invention relates to any one of the aforementioned formulations, wherein the propellant is a hydrofluorocarbon propellant.

In certain embodiments, the invention relates to any one of the aforementioned formulations, wherein the inert gas is argon.

### Exemplary Methods of the Invention

In certain embodiments, the invention relates to a method for enhancing the bioavailability of a corticosteroid from an oil-in-water emulsion, comprising the step of varying the concentrations of surfactants, co-surfactants, emollients and water, thereby forming an improved corticosteroid-containing emulsion.

In certain embodiments, the invention relates to a method for enhancing the bioavailability of a corticosteroid from a formulation, comprising the step of varying the concentrations of surfactants, co-surfactants, emollients and water, thereby forming an improved corticosteroid-containing formulation.

In certain embodiments, the invention relates to any one of the aforementioned methods, wherein the improved corticosteroid-containing emulsion or the improved corticosteroid-containing formulation is intended for topical administration.

In certain embodiments, the invention relates to any one of the aforementioned methods, wherein the improved corticosteroid-containing emulsion is any one of the aforementioned emulsions.

In certain embodiments, the invention relates to any one of the aforementioned methods, wherein the improved corticosteroid-containing formulation is any one of the aforementioned formulations.

In certain embodiments, the invention relates to any one of the aforementioned methods, wherein improved corticosteroid-containing emulsions containing 0.15% hydrocortisone butyrate reduce the total body surface area presenting with atopic dermatitis to a greater than extent than do emulsions containing 0.1% hydrocortisone butyrate at Day 8.

In certain embodiments, the invention relates to any one of the aforementioned methods, wherein the concentration of hydrocortisone 17-butyrate is 0.15% by weight of the improved corticosteroid-containing emulsion; and the improved corticosteroid-containing emulsion reduces the total body surface area presenting with atopic dermatitis to a greater than extent than do emulsions containing 0.1% hydrocortisone butyrate at Day 8.

In certain embodiments, the invention relates to any one of the aforementioned methods, wherein the concentration of hydrocortisone 17-butyrate is 0.15% by weight of the improved corticosteroid-containing emulsion; and the improved corticosteroid-containing emulsion reduces the total body surface area presenting with atopic dermatitis to a greater than extent than do emulsions containing 0.1% hydrocortisone butyrate at Day 15.

In certain embodiments, the invention relates to any one of the aforementioned methods, wherein the reduction in Lichenification symptoms from improved corticosteroid-containing emulsions exhibits a dose response to hydrocortisone butyrate concentration at Day 29.

In certain embodiments, the invention relates to any one of the aforementioned methods, wherein the reduction in Excoriation symptoms from improved corticosteroid-containing emulsions exhibits a dose response to hydrocortisone butyrate concentration at Day 29.

In certain embodiments, the invention relates to any one of the aforementioned methods, wherein the reduction in Oozing/Crusting symptoms from improved corticosteroid-containing emulsions exhibits a dose response to hydrocortisone butyrate concentration at Day 15.

In certain embodiments, the invention relates to any one of the aforementioned methods, wherein the reduction in Induration/Papulation symptoms from improved corticosteroid-containing emulsions exhibits a dose response to hydrocortisone butyrate concentration at Day 15.

### Exemplary Properties of Emulsions and Formulations of the Invention

In certain embodiments, the invention relates to any one of the aforementioned emulsions, wherein the emulsion is a cream or a lotion.

In certain embodiments, the invention relates to any one of the aforementioned formulations, wherein the formulation forms a foam.

In certain embodiments, the invention relates to any one of the aforementioned emulsions or formulations that, upon application to the skin of an affected subject, is non-irritating.

In certain embodiments, the invention relates to any one of the aforementioned emulsions or formulations that, upon application to the skin of an affected subject, is well-tolerated.

In certain embodiments, the invention relates to any one of the aforementioned emulsions or formulations that, upon application to the skin of an affected subject, is non-cytotoxic.

In certain embodiments, the invention relates to any one of the aforementioned emulsions or formulations that, upon application to the skin of an affected subject, is weakly sensitizing. In certain embodiments, the invention relates to any one of the aforementioned emulsions or formulations that, upon application to the skin of an affected subject, is non-sensitizing.

In certain embodiments, the invention relates to any one of the aforementioned emulsions or formulations that, upon application to the skin of an affected subject, does not produce edema or erythema.

In certain embodiments, the invention relates to any one of the aforementioned emulsions or formulations that, upon application to the skin of an affected subject, moisturizes the skin.

In certain embodiments, the invention relates to any one of the aforementioned emulsions or formulations that, upon application to the skin of an affected subject, increases hydration of the skin.

In certain embodiments, the invention relates to any one of the aforementioned emulsions or formulations that, upon application to the skin of an affected subject, reduces transepidermal water loss.

In certain embodiments, the invention relates to any one of the aforementioned emulsions or formulations that, upon application to the skin of an affected subject, improves bioavailability of the corticosteroid as compared to a reference emulsion or formulation.

In certain embodiments, the invention relates to any one of the aforementioned emulsions or formulations that, upon application to the skin of an affected subject, releases a larger quantity of the corticosteroid over time as compared to a reference formulation.

In certain embodiments, the reference emulsion or formulation comprises less water by weight. In certain embodiments, the reference emulsion or formulation comprises less cetostearyl alcohol by weight. In certain embodiments, the reference emulsion or formulation comprises more dimethicone by weight. In certain embodiments, the reference emulsion or formulation comprises more vegetable oil by weight. In certain embodiments, the reference emulsion or formulation comprises more white petrolatum by weight. In certain embodiments, the reference emulsion or formulation comprises more mineral oil by weight. In certain embodiments, the reference emulsion or formulation comprises steareth-10.

In certain embodiments, the invention relates to any one of the aforementioned emulsions or formulations, wherein, under standard conditions, the rate of release of the corticosteroid at 6 hours is about 2 to about 6 μg/cm²/hr. In certain embodiments, the invention relates to any one of the aforementioned emulsions or formulations, wherein, under standard conditions, the rate of release of the corticosteroid at 6 hours is about 2.5 to about 4.5 μg/cm²/hr. In certain embodiments, the invention relates to any one of the aforementioned emulsions or formulations, wherein, under standard conditions, the rate of release of the corticosteroid at 6 hours is about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0 μg/cm²/hr. In certain embodiments, standard conditions are the conditions described in Example 3.

In certain embodiments, the invention relates to any one of the aforementioned oil-in-water emulsions, wherein the viscosity of the oil-in-water emulsion is about 55,000 to about 110,000 cP. In certain embodiments, the invention relates to any one of the aforementioned oil-in-water emulsions, wherein the viscosity of the oil-in-water emulsion is about 55,000, about 60,000, about 65,000, about 70,000, about 75,000, about 80,000, about 85,000, about 90,000, about 95,000, about 100,000, about 105,000, or about 110,000 cP.

In certain embodiments, the invention relates to any one of the aforementioned emulsions, wherein the mean VCA score of the emulsion is about 0.9 to about 1.5. In certain embodiments, the invention relates to any one of the aforementioned emulsions, wherein the mean VCA score of the emulsion is about 0.9, about 1.0, about 1.1, about 1.2, about 1.3, about 1.4, or about 1.5.

In certain embodiments, the invention relates to any one of the aforementioned formulations, wherein the density of the dispensed foam is about 0.13 to about 0.50 g/cm³. In certain embodiments, the invention relates to any one of the aforementioned formulations, wherein the density of the dispensed foam is about 0.13, about 0.14, about 0.15, about 0.16, about 0.17, about 0.18, about 0.19, about 0.20, about 0.21, about 0.22, about 0.23, about 0.24, about 0.25, about 0.26, about 0.27, about 0.28, about 0.29, or about 0.30 g/cm³.

In certain embodiments, the invention relates to any one of the aforementioned formulations, wherein the mean VCA score of the dispensed foam is about 0.9 to about 1.5. In certain embodiments, the invention relates to any one of the aforementioned formulations, wherein the mean VCA score of the dispensed foam is about 0.9, about 1.0, about 1.1, about 1.2, about 1.3, about 1.4, or about 1.5.

In certain embodiments, the invention relates to any one of the aforementioned emulsions, wherein improved corticosteroid-containing emulsions containing 0.15% hydrocortisone butyrate reduce the total body surface area presenting with atopic dermatitis to a greater than extent than do emulsions containing 0.1% hydrocortisone butyrate at Day 8.

In certain embodiments, the invention relates to any one of the aforementioned emulsions, wherein the concentration of hydrocortisone 17-butyrate is 0.15% by weight of the improved corticosteroid-containing emulsion; and the improved corticosteroid-containing emulsion reduces the total body surface area presenting with atopic dermatitis to a greater than extent than do emulsions containing 0.1% hydrocortisone butyrate at Day 8.

In certain embodiments, the invention relates to any one of the aforementioned emulsions, wherein the concentration of hydrocortisone 17-butyrate is 0.15% by weight of the improved corticosteroid-containing emulsion; and the improved corticosteroid-containing emulsion reduces the total body surface area presenting with atopic dermatitis to a greater than extent than do emulsions containing 0.1% hydrocortisone butyrate at Day 15.

In certain embodiments, the invention relates to any one of the aforementioned emulsions, wherein the reduction in Lichenification symptoms from improved corticosteroid-containing emulsions exhibits a dose response to hydrocortisone butyrate concentration at Day 29.

In certain embodiments, the invention relates to any one of the aforementioned emulsions, wherein the reduction in Excoriation symptoms from improved corticosteroid-containing emulsions exhibits a dose response to hydrocortisone butyrate concentration at Day 29.

In certain embodiments, the invention relates to any one of the aforementioned emulsions, wherein the reduction in Oozing/Crusting symptoms from improved corticosteroid-containing emulsions exhibits a dose response to hydrocortisone butyrate concentration at Day 15.

In certain embodiments, the invention relates to any one of the aforementioned emulsions, wherein the reduction in Induration/Papulation symptoms from improved corticosteroid-containing emulsions exhibits a dose response to hydrocortisone butyrate concentration at Day 15.

### Exemplary Emulsions or Formulations of the Invention for Particular Uses

In certain embodiments, the invention relates to any one of the aforementioned emulsions or formulations for use in the treatment of a skin disorder.

In certain embodiments, the invention relates to any one of the aforementioned emulsions or formulations, wherein the skin disorder is a dermatosis.

In certain embodiments, the invention relates to any one of the aforementioned emulsions or formulations, wherein the skin disorder is seborrheic dermatitis.

In certain embodiments, the invention relates to any one of the aforementioned emulsions or formulations, wherein the skin disorder is atopic dermatitis.

### Exemplary Methods of Use

In certain embodiments, the invention relates to a method of treating a skin disorder, comprising the step of:

applying topically to an area of skin of a subject in need thereof a therapeutically-effective amount of any one of the aforementioned emulsions or formulations.

In certain embodiments, the invention relates to any one of the aforementioned methods, wherein the emulsion or the formulation is applied once daily or twice daily.

In certain embodiments, the invention relates to any one of the aforementioned methods, wherein the subject is human.

In certain embodiments, the invention relates to any one of the aforementioned methods, wherein the skin disorder is a dermatosis.

In certain embodiments, the invention relates to any one of the aforementioned methods, wherein the skin disorder is seborrheic dermatitis.

In certain embodiments, the invention relates to any one of the aforementioned methods, wherein the skin disorder is atopic dermatitis.

In certain embodiments, the invention relates to any one of the aforementioned methods, wherein improved corticosteroid-containing emulsions containing 0.15% hydrocortisone butyrate reduce the total body surface area presenting with atopic dermatitis to a greater than extent than do emulsions containing 0.1% hydrocortisone butyrate at Day 8.

In certain embodiments, the invention relates to any one of the aforementioned methods, wherein the concentration of hydrocortisone 17-butyrate is 0.15% by weight of the improved corticosteroid-containing emulsion; and the improved corticosteroid-containing emulsion reduces the total body surface area presenting with atopic dermatitis to a greater than extent than do emulsions containing 0.1% hydrocortisone butyrate at Day 8.

In certain embodiments, the invention relates to any one of the aforementioned methods, wherein the concentration of hydrocortisone 17-butyrate is 0.15% by weight of the improved corticosteroid-containing emulsion; and the improved corticosteroid-containing emulsion reduces the total body surface area presenting with atopic dermatitis to a greater than extent than do emulsions containing 0.1% hydrocortisone butyrate at Day 15.

In certain embodiments, the invention relates to any one of the aforementioned methods, wherein the reduction in Lichenification symptoms from improved corticosteroid-containing emulsions exhibits a dose response to hydrocortisone butyrate concentration at Day 29.

In certain embodiments, the invention relates to any one of the aforementioned methods, wherein the reduction in Excoriation symptoms from improved corticosteroid-containing emulsions exhibits a dose response to hydrocortisone butyrate concentration at Day 29.

In certain embodiments, the invention relates to any one of the aforementioned methods, wherein the reduction in Oozing/Crusting symptoms from improved corticosteroid-containing emulsions exhibits a dose response to hydrocortisone butyrate concentration at Day 15.

In certain embodiments, the invention relates to any one of the aforementioned methods, wherein the reduction in Induration/Papulation symptoms from improved corticosteroid-containing emulsions exhibits a dose response to hydrocortisone butyrate concentration at Day 15.

### EXEMPLIFICATION

The invention now being generally described, it will be more readily understood by reference to the following examples which are included merely for purposes of illustration of certain aspects and embodiments of the present invention, and are not intended to limit the invention.

### Example 1

### Compositions and Method of Manufacture

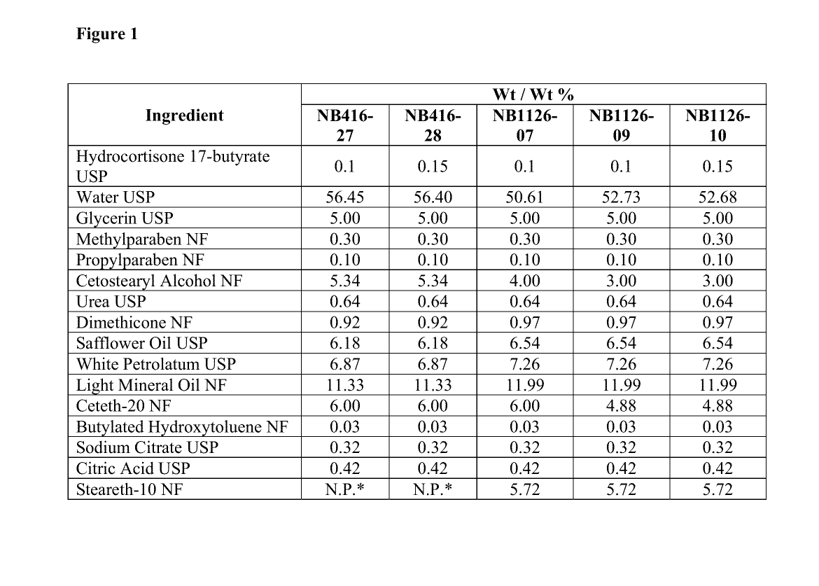

An example product concentrate (NB416-27; see FIG. 1) was manufactured by the procedure outlined below:

### Part A: Oil Phase Preparation

1. Charge Ceteth-20 (I) light mineral oil, white petrolatum, dimethicone, safflower oil, butylated hydroxytoluene and cetostearyl alcohol into a Stainless Steel tank and heat until fully melted.

### Part B: Aqueous Phase Preparation

1. Charge Purified Water (I) and Glycerin into a Stainless Steel tank and heat to 75-80° C.
2. Charge and dissolve citric acid (I) and sodium citrate (I) as well as urea, methyl paraben and propyl paraben while mixing.
3. Continue mixing until a clear solution is obtained while maintaining a temperature of 65-95° C.

### Part C: Drug Phase Preparation

1. Charge a Stainless Steel tank with Purified Water (II), citric acid (II), sodium citrate (II) and ceteth-20 (II).
2. Mix slowly at room temperature to dissolve.
3. Add hydrocortisone butyrate and mix until fully wetted and dispersed.

### Part D: Drug Product Concentrate Formation

1. Add Part A to Part B while high shear mixing at 65-95° C.
2. Cool the emulsion with an outside cold water jacket to below 50° C. while high shear mixing.
3. Discontinue high shear mixing. Start low shear mixing and continue cooling with cold water jacket to form the vehicle emulsion.
4. When the temperature of the vehicle emulsion is below 37° C., add Part C and continue mixing until uniform.
5. Cool to room temperature. Adjust to final volume with DI water. Mix until uniform.

Following manufacturing of the Drug Product and Vehicle Concentrate, the finished Drug Product and Drug Product Vehicle is produced as outlined below.
1. Aerosol cans are cleaned with compressed air and vacuum.
2. Product Concentrate is filled into cans.
3. Valves are placed onto the cans.
4. Cans are crimped and hydrofluorocarbon propellant is charged.
5. The aerosol can valve and dip-tube is purged with argon gas.

Propellant concentrations range from 8 to 15% by weight of packaged product, argon concentrations range from 0.8 to 4.0% by weight of packaged product.

### Example 2

### Vasoconstriction Assay Results

When applied to the skin, topical corticosteroids produce a localized skin-blanching response (vasoconstriction) due to the constriction of the superficial blood vessels of the skin. The degree of blanching assessed by visual scoring is a measure of the inherent potency of the drug and its capacity to diffuse through the stratum corneum. The vasoconstrictor assay (VCA) is the most widely used surrogate test to assess the potency of topical corticosteroids, and has been shown to correlate reasonably well with the clinical efficacy of corticosteroid formulations, although it is not the mechanism by which efficacy is obtained (i.e., efficacy is a function of the drug's anti-inflammatory, immunosuppressive, or anti-mitotic properties). The results of the vasoconstrictor assay have been used to a) classify topical corticosteroids into seven potency classes (Class 1 through 7); and b) identify and optimize new formulations for clinical development.

A single point, randomized, evaluator blinded, within subject, single center study was conducted in 37 subjects.

Healthy, adult volunteer subjects provided written informed consent and were screened for the study. Subjects meeting the entry criteria were scheduled for the two-day study period. Scheduling was arranged so that the timing of the application of test articles and study evaluations was “staggered” to accommodate the clinical evaluations of multiple subjects. On Day 1, eight ˜1 cm² test sites were identified on the ventral forearms of subjects (4 test sites on one forearm and 4 test sites on the other forearm) and marked with an indelible pen. A single application of approximately 10 mg of each test article was made to the designated test site in accordance with a computer-generated randomization code, thus blinding the evaluator to the application sequence. Five foam formulations and three reference topical steroid products were evaluated. All of the test articles were applied later in the afternoon (e.g., at approximately 4:00 pm) on Day 1, after which the test sites on each arm were protected using a raised perforated guard. The guards were secured to the arms with a non-occlusive tape, and the subjects were scheduled to return the following day (18 hours after test article application) after being instructed to keep the test sites dry for 16 hours after test article application. After 16 hours, the subject was instructed to remove the protective guards, and gently wash the test sites with mild soap and water.

Upon return to the clinic two hours later (18 hours after the test article applications or at 10:00 am based upon a 4:00 pm application time on Day 1), an experienced evaluator performed the visual assessment of vasoconstriction (skin-blanching) based on a four-point scale (0-3). This was the final clinical evaluation. Subjects were dismissed from the study following this evaluation. All subjects who returned to the clinic as instructed completed the study.

The primary efficacy measurement was the amount of skin blanching (vasoconstriction) assessed visually approximately 18 (±1) hours after the end of the test article applications. The degree of skin blanching was assessed visually using the following four-point ordinal scale:

0=No blanching; no change from surrounding area.

1=Mild blanching; slight or indistinct outline at application site.

2=Moderate blanching; discernible outline at application site.

3=Marked blanching; distinct outline at application site.

All subjects were classified into the intent-to-treat (ITT) and per-protocol (PP) populations according to the following definitions. The ITT population was defined as all subjects who were randomized and had at least one test article applied. All efficacy and safety data were summarized using the ITT population. The PP population was a subset of the ITT population consisting of subjects without significant protocol violations, who had test articles applied, and completed the vasoconstriction assessment as described in the protocol.

All statistical tests were performed at a significance level of 5% (two-tailed). The analysis of efficacy was conducted on both the ITT and PP populations with the ITT population considered as the primary population for statistical analysis.

The primary analysis tests the null hypothesis that the visually assessed treatment blanching score means are equal to each other. Since this is a within-subject design, the visual skin blanching data was analyzed for mean differences among treatments using a randomized blocks analysis of variance (ANOVA) and a nonparametric analog using the ranks of the scores with subject as the blocking variable.

Within this analysis, pairwise comparisons of the mean visual assessment scores were performed using the Ryan-Einot-Gabriel-Welsch Multiple Range Test (REGWQ) which controls the experiment wise Type I error rate at 5% under the complete null hypothesis.

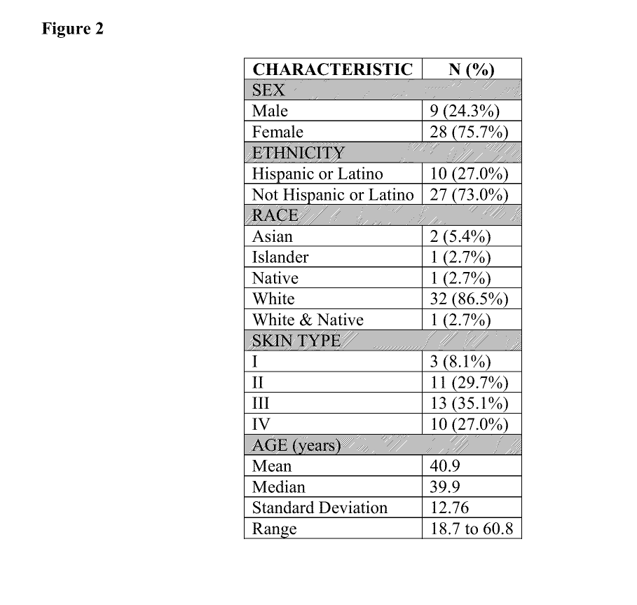

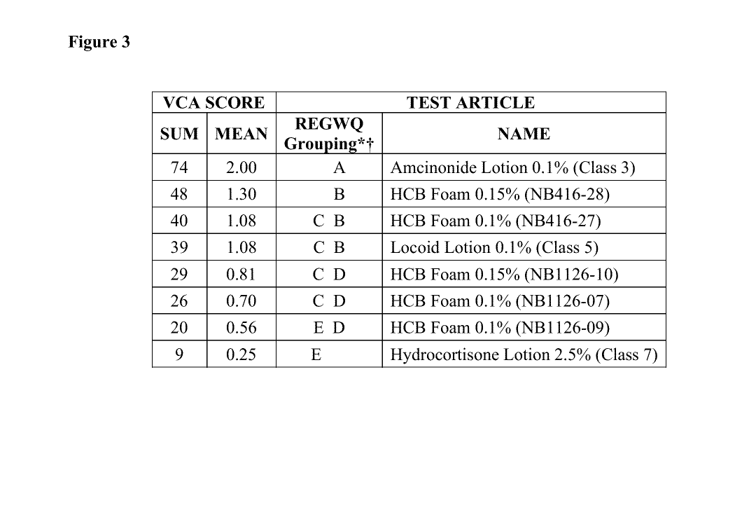

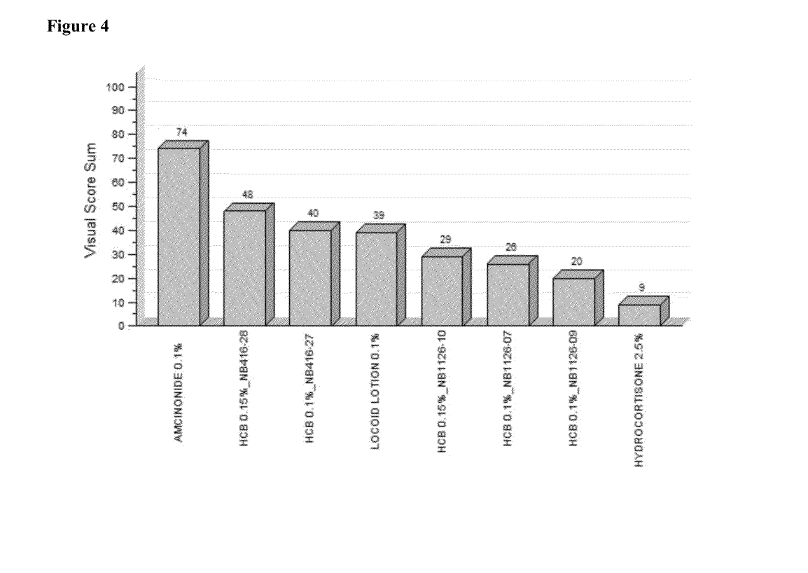

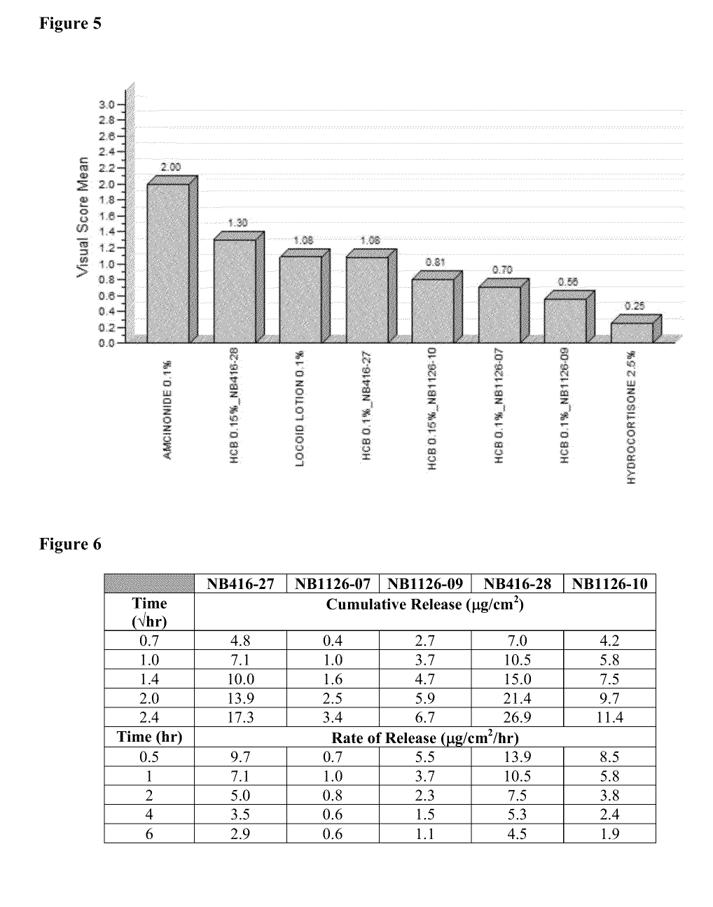

See FIG. 2, FIG. 3, FIG. 4, and FIG. 5.

### Example 3

### In Vitro Release Kinetics

In order for topically applied drug products to be effective the constituent drug substance must be released from the vehicle before it can traverse to the stratum corneum. Although not directly correlated to in vivo bioavailability, characterization of drug product release profiles allows for the identification of formulation with the potential to effect drug product bioavailability. A Franz vertical diffusion cell was used to examine the rate and extent of API release from foam concentrates in vitro. The experimental conditions were as below.
Instrument: Logan Instruments Corp System 912-12
Membrane: Whatman, PTFE, 5.0 um, 37 mm
Temperature: 32.5° C.
Speed: 300 rpm
Time pulls (min): 30, 60, 120, 240, and 360
Media: For Base Line Conditions: 70% Buffer, 30% Ethanol
1) Turn all parts of the instrument.
2) Prime, fill and drain sample cells three times
3) Fill the media reservoir with the appropriate media and repeat step 2.
4) Prepare the cells:
   a. Place the membrane on top of the cell, place the cap, and then clap them down together.
   b. Fill cells with media.
   c. Transfer the sample via direct transfer. Make sure to take an initial weight and a final weight after filling every test article.
5) Collect samples
   a. Set flush volume to 1.5 mL
   b. Set media replace volume to 4.6 mL with return to cell
   c. Set waste to 1.5 mL
   d. Set sample to 1.5 mL
   e. Set sampling time intervals
6) Measure hydrocortisone butyrate concentration in samples via HPLC
   Instrument: Liquid Chromatograph equipped with a UV Detector
   Column: Zorbax™ SB-CN Dimensions: 150×4.6 mm, 3.5 μm, Agilent® Part Number 863953-905 or equivalent
   Mobile Phase A Composition: 5 mM Phosphate Buffer pH 4.8
   Mobile Phase B Composition: Methanol
   Mobile Phase C Composition: Acetonitrile

Mobile Phase Composition Table:

| Mobile Phase A % | Mobile Phase B % | Mobile Phase C % |
|---|---|---|
| 60 | 20 | 20 |

Column Temperature: 40° C.
Flow Rate: 1.2 mL/min
Detection: UV at 245 nm
Injection Volume: 25 μL
Run Time: 20 min

1) Sample Preparation

I. Foam Sample:
a. Load autosampler with HPLC sample vials
b. Fill chambers with
c. Dispense 10-15 grams drug product concentrate
d. Fill a syringe with sample.
e. Tare a balance, place the syringe containing the sample on the balance, and record the weight.
f. Slowly add approximately 0.8-1.0 g of sample into cell chamber #1 (ensure that the sample fills the cell chamber, avoid creating air gaps or headspace between the sample and filter).
g. Place the syringe back onto the tared balance and record the weight in grams, record the weight (Back weighing).
h. Continue with steps g through h until all sample cell chambers are full.

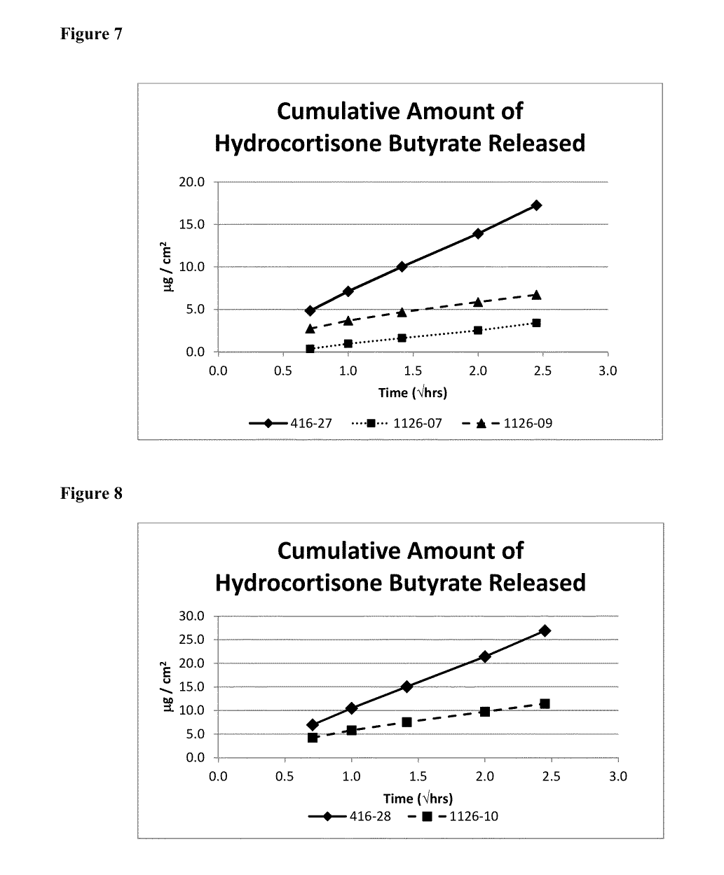

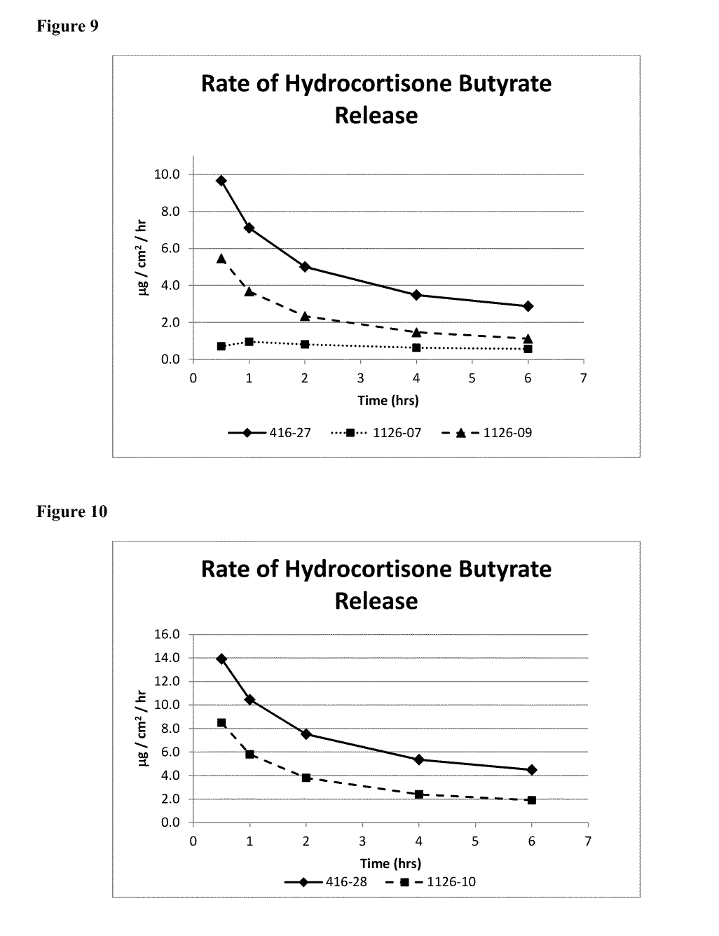

See FIG. 6, FIG. 7, FIG. 8, FIG. 9, and FIG. 10.

### Example 4

### Product Densities

When dispensed from an aerosol can, the compositions of the method form a time- and temperature-stable low density foam. The densities of dispensed foam compositions were measured as follows.

Product was dispensed into a receptacle of known weight and volume. The product was dispensed into the receptacle so that there were no voids. Excess material was removed from the top of the receptacle. The mass of the test article and receptacle is determined with the test article density calculated using the formula:

Density=(MASS_{T}−MASS_{R})/VOLUME_{R}

### Where:

MASS_{T}=total mass of test article and receptacle
MASS_{R}=mass of receptacle
VOLUME_{R}=volume of receptacle

See FIG. 11.

### Example 5

### Product Viscosities

Aerosol foam concentrate viscosity affects dispensing from the can and the rate of diffusion of active ingredients in the foam vehicle. The viscosities of foam concentrates of the method were determined by the procedure below.
1. Turn on the water bath and set it to 25° C. Allow the temperature to stabilize at 25° C. for approximately 5 minutes.
2. Calibrate the viscometer with a 12,500 cP standard using spindle #25, speed 12 rpm at 25° C.
3. Remove sample jacket attachment from pivot cup. Clamp sample jacket at appropriate level based on sample length.
4. Set up the Helipath™ Stand and T-bar spindle S96.
6. Turn on the Brookfield DV-I Viscometer and Auto zero the viscometer.
7. Set the speed to 1.5 rpm or 3.0 rpm.
8. Transfer approximately 10 mL of sample into the sample chamber, by cutting approximately 2 inches from the pipet tip of a disposable pipet. The sample should be slowly pulled into the cut pipet and dispensed into the Brookfield sample vessel, filling from bottom to top with minimal disturbance and tapping.
9. Place the sample chamber into water jacket.
10. Put the spindle into the sample.
11. Allow the sample temperature to equilibrate for 30 minutes. DO NOT allow the spindle to turn during the equilibration time. The motor should be off
12. After the equilibration time, select the Time Stop test method.
13. Set time to 5 minutes.
14. Start the method.
15. After 5 minutes, the viscosity reading will be displayed. Record the viscosity.
16. Repeat the Time Stop measurement two additional times.
17. Calculate the average of the three measurements and report the average.

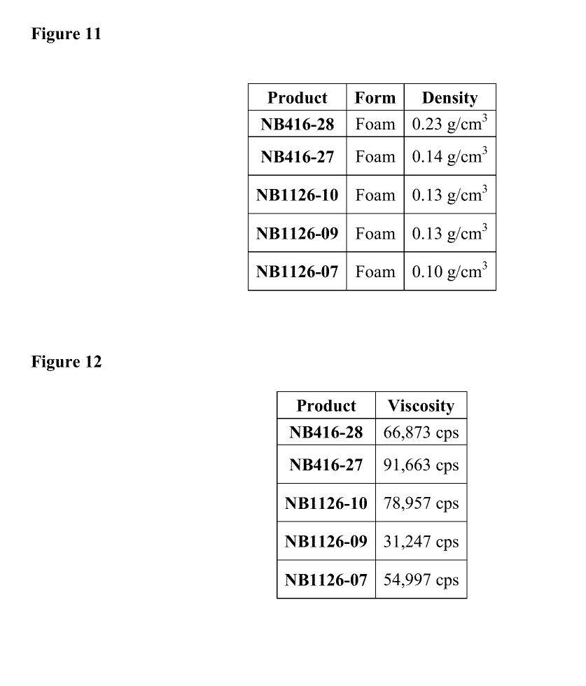

See FIG. 12.

### Example 6

### Efficacy Trial Results

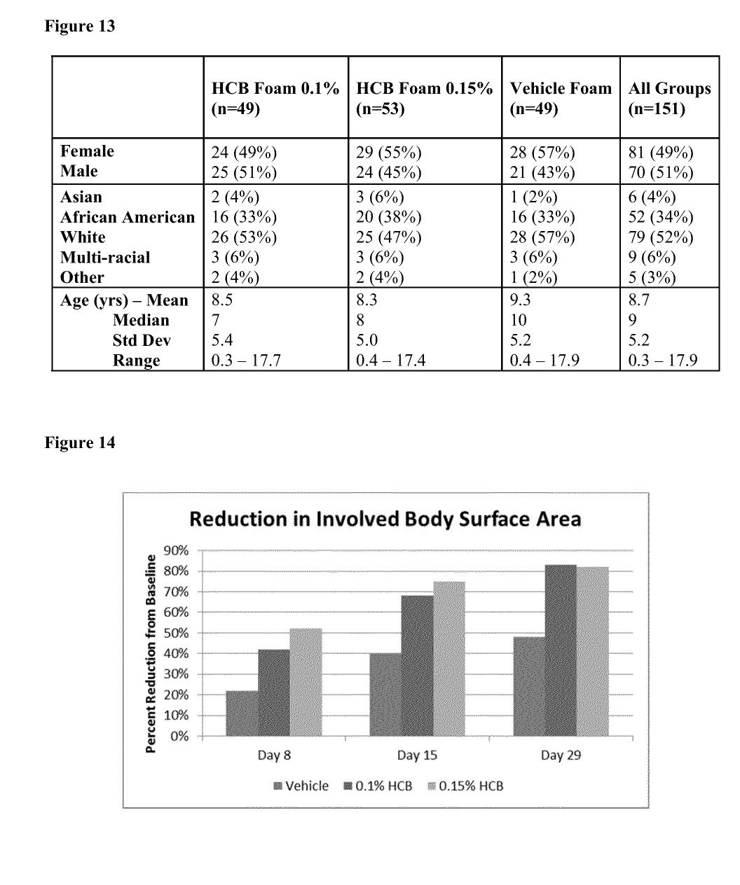

A multicenter, randomized, double-blinded, vehicle-controlled, parallel group evaluation of twice daily 0.1% Hydrocortisone Butyrate Foam in comparison to 0.15% Hydrocortisone Butyrate Foam in the treatment of mild to moderate Atopic Dermatitis in pediatric subjects 3 months to 18 years of age was conducted in 151 subjects (FIG. 13).

Male and female subjects in general good health and presenting with a clinical diagnosis of stable mild to moderate Atopic Dermatitis on no less than 10% of the body surface area, and having a severity of 2 or 3 on the Investigators Global Assessment scale, were enrolled in the trial. All subjects were 3 months to less than 18 years of age (at least 3 months old but not yet reached their 18^{th} birthday at the time of enrollment) at enrollment, and each subject's parent/legal guardian read, understood and signed a written, IRB-approved, informed consent prior to admission into the study. Additionally all subjects between 7 and 17 years of age, inclusive, provided written assent prior to admission to the study.

At the initial screening/baseline visit (Day 1), study procedures were explained and an informed consent signed. Consenting subjects underwent a medical and dermatologic history and concomitant medication review. Subjects underwent a limited physical examination including vital signs, clinical evaluations and an inclusion/exclusion (I/E) criteria review to determine subject eligibility. All female subjects age 10 and above had a negative urine pregnancy test. Enrolled subjects had blood and urine collected for routine safety labs (chemistry, hematology and urinalysis) and were randomized to one of the four Treatment Groups. Test article was dispensed, and subject diaries and subject instructions for use were provided. The first dose of test article application was applied in the clinic under staff supervision. Subjects were instructed to apply the assigned study medication twice daily to all affected areas and follow-up office visits were scheduled.

At the follow-up visits on days 8, 15 and 22, subjects were interviewed for possible adverse events and any changes in concomitant medications. Clinical evaluations and subject assessments were performed at Day 8 and Day 22. Subject diaries were reviewed/collected and new diaries dispensed. Test articles were dispensed and/or returned as necessary and instructions for use were reviewed.

At the end of study visit (Day 29), subjects were interviewed for possible adverse events and any changes in concomitant medications and urine pregnancy tests were performed as appropriate. Clinical evaluations per protocol and subject assessments were performed and subjects were asked to evaluate changes in their atopic dermatitis compared to baseline. Blood and urine samples were collected for safety laboratory tests and study diaries were collected and reviewed. All test articles were collected.

Throughout the study, the Investigator's Global Assessment (IGA), and evaluations of clinical signs, and local skin reactions (LSRs) were performed by the same investigator/evaluator for a given subject.

Efficacy was assessed by the investigator or their designee, with the exclusion of pruritus and the subject global assessment score which are performed by the subject, as follows:

The Investigator's Global Assessment (IGA) of overall severity of atopic dermatitis used a 5-point ordinal scale from 0=clear to 4=severe. This is a static morphological scale referred to a point in time and not a comparison to previous visits.

Investigator's Global Assessment (IGA)

| SCORE | CATEGORY | DEFINITION |
|---|---|---|
| 0 | Clear | No signs of inflammatory AD |
| 1 | Almost Clear | Faint, barely detectable erythema and/or trace |
| | | residual elevation in limited areas; neither |
| | | excoriation nor oozing/crusting are present |
| 2 | Mild | Light pink erythema and slightly perceptible |
| | | elevation; excoriation, if present, is mild |
| 3 | Moderate | Dull red, clearly distinguishable erythema and |
| | | clearly perceptible elevation but not extensive; |
| | | excoriation or oozing/crusting, if present, are |
| | | mild to moderate. |
| 4 | Severe | Deep/dark red erythema, and marked and |
| | | extensive elevation; excoriation and oozing/ |
| | | crusting are present. |

Assessments of the following Clinical Signs & Symptoms of Atopic Dermatitis were performed.

Erythema (E)

| | | |
|---|---|---|
| 0 | None | None |
| 1 | Mild | Faintly detectable erythema: very light pink |
| 2 | Moderate | Dull red, clearly distinguishable |
| 3 | Severe | Deep/dark red |

Induration/Papulation (I/P)

| | | |
|---|---|---|
| 0 | None | None |
| 1 | Mild | Barely perceptible elevation |
| 2 | Moderate | Clearly perceptible elevation but not extensive |
| 3 | Severe | Marked and extensive elevation |

Excoriations (Ex)

| | | |
|---|---|---|
| 0 | None | None |
| 1 | Mild | Scant evidence of excoriations with no signs of deeper |
| | | skin damage (Erosion, crust) |
| 2 | Moderate | Several Linear Marks of skin with some showing |
| | | evidence of deeper skin injury (erosion, crust) |
| 3 | Severe | Many erosive or crusty lesions |

Lichenification (L)

| | | |
|---|---|---|
| 0 | None | None |
| 1 | Mild | Slight thickening of the skin discernible only by touch |
| | | and with skin markings minimally exaggerated. |
| 2 | Moderate | Definite thickening of the skin with skin marking |
| | | exaggerated so that they form a visible criss-cross |
| | | pattern. |
| 3 | Severe | Thickened indurated skin with skin markings visibly |
| | | portraying an exaggerated criss-cross pattern. |

Oozing/Crusting (O)

| | | |
|---|---|---|
| 0 | None | None |
| 1 | Mild | Evidence of exudation |
| 2 | Moderate | Serous brown, yellow or green, exudations and/or drying |
| | | of the discharge. |
| 3 | Severe | Many dry scabs and/or exudations. |

Pruritus - itching (I) - performed by subject*
considering symptoms over past 24 hours

| | | |
|---|---|---|
| 0 | None | None |
| 1 | Mild | Occasional, slight itching/scratching |
| 2 | Moderate | Constant or intermittent itching/scratching/discomfort |
| | | which is not disturbing sleep. |
| 3 | Severe | Bothersome itching/scratching/discomfort which is |
| | | disturbing sleep |

On the Day 29 visit, the subject* evaluated his/her atopic dermatitis as compared to his/her atopic dermatitis condition at Baseline, according to the following scale:

Subject's AD Improvement Assessment

| | |
|---|---|
| 1 | Excellent improvement |
| 2 | Good improvement |
| 3 | Moderate improvement |
| 4 | No improvement |
| 5 | Worse |

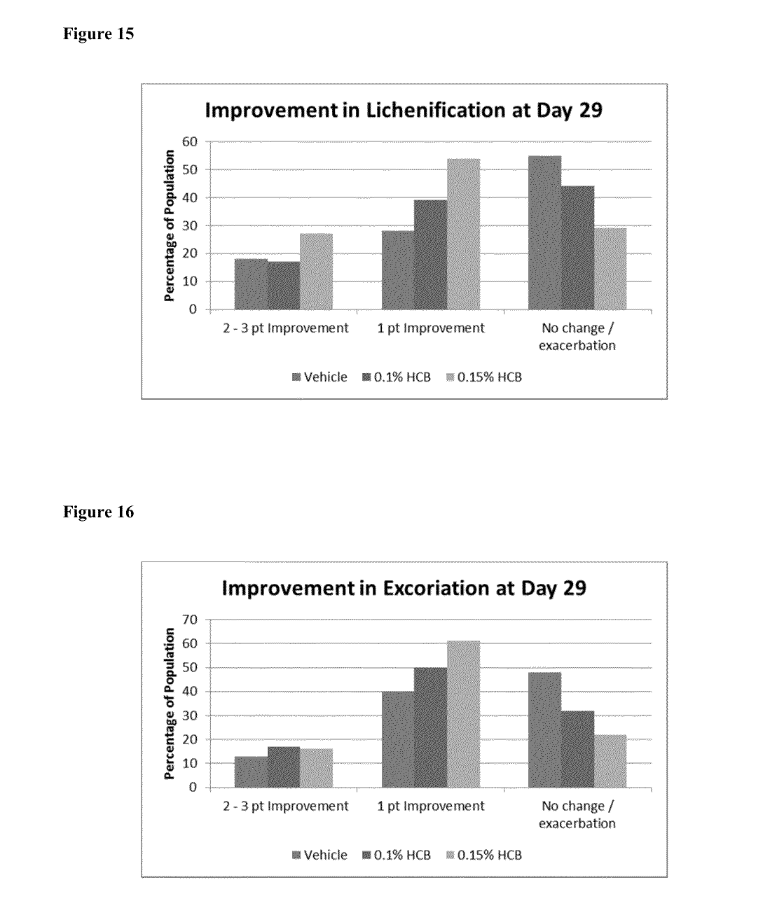

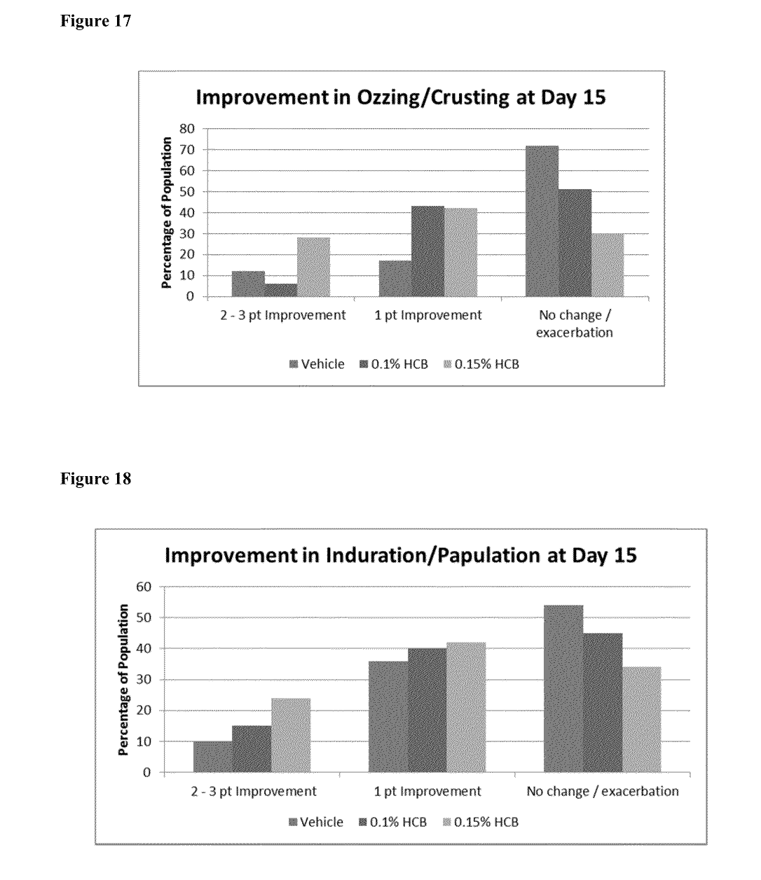

*For younger subjects the parent or guardian performed the assessment on behalf of the subject.
See FIG. 14, FIG. 15, FIG. 16, and FIG. 17, and FIG. 18.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method for enhancing the bioavailability of a corticosteroid from an oil-in-water emulsion, comprising the step of varying the concentrations of surfactants, co-surfactants, emollients and water, thereby forming an improved corticosteroid-containing emulsion.

2. The method of claim 1, wherein the improved corticosteroid-containing emulsion comprises
a corticosteroid;
a surfactant and a co-surfactant;
an oil phase comprising at least a first emollient and a second emollient; and
water;
wherein the first emollient is a vegetable oil and the second emollient is a mineral oil; and
the weight ratio of vegetable oil-to-mineral oil is about 0.03 to about 1.00.

3. The method of claim 2, wherein the total concentration of surfactants and co-surfactants in the improved corticosteroid-containing emulsion is about 8.0% to about 12.0% by weight of the improved corticosteroid-containing emulsion.

4. The method of claim 2, wherein the surfactant is ceteth-20; and the co-surfactant is cetostearyl alcohol.

5. The method of claim 2, wherein the improved corticosteroid-containing emulsion does not comprise steareth-10.

6. The method of claim 2, wherein the emollients are safflower oil, dimethicone, light mineral oil, and white petrolatum.

7. The method of claim 2, wherein the corticosteroid is hydrocortisone butyrate.

8. The method of claim 7, wherein the hydrocortisone butyrate is hydrocortisone 17-butyrate.

9. The method of claim 8, wherein the concentration of hydrocortisone-17 butyrate is about 0.1% by weight of the improved corticosteroid-containing emulsion.

10. The method of claim 8, wherein the concentration of hydrocortisone 17-butyrate is 0.15% by weight of the improved corticosteroid-containing emulsion.

11. The method of claim 2, wherein the vegetable oil is safflower oil, sunflower oil, corn oil, sesame oil, peanut oil, canola oil, or olive oil.

12. The method of claim 2, wherein the vegetable oil is safflower oil.

13. The method of claim 2, wherein the mineral oil is light mineral oil.

14. The method of claim 1, wherein the improved corticosteroid-containing emulsion consists essentially of, by weight of the oil-in-water emulsion

| | |
|---|---|
| Hydrocortisone 17-butyrate USP | From about 0.01% to about 0.25% |
| Water USP | From about 30% to about 80% |
| Glycerin USP | From about 2.5% to about 7.5% |
| Methylparaben NF | From about 0.15% to about 0.45% |
| Propylparaben NF | From about 0.05% to about 0.15% |
| Cetostearyl Alcohol NF | From about 2.5% to about 7.5% |
| Urea USP | From about 0.3% to about 0.9% |
| Dimethicone NF | From about 0.5% to about 1.5% |
| Safflower Oil USP | From about 3.0% to about 9.0% |
| White Petrolatum USP | From about 3.0% to about 9.0% |
| Light Mineral Oil NF | From about 6.0% to about 18.0% |
| Ceteth-20 NF | From about 3.0% to about 9.0% |
| Butylated Hydroxytoluene NF | From about 0.015% to about 0.045% |
| Sodium Citrate USP | From about 0.15% to about 0.45% |
| Citric Acid USP | From about 0.20% to about 0.60% |

15. A method of treating a skin disorder, comprising the step of:
applying topically to an area of skin of a subject in need thereof a therapeutically-effective amount of an improved corticosteroid-containing emulsion of claim 1.

16. The method of claim 15, wherein the improved corticosteroid-containing emulsion or the formulation is applied once daily or twice daily.

17. The method of claim 15, wherein the subject is human.

18. The method of claim 15, wherein the skin disorder is a dermatosis.

19. The method of claim 15, wherein the skin disorder is atopic dermatitis.
